(19) United States
(12) Patent Application Publication
(10) Pub. No.: US 2025/0340715 A1
(43) Pub. Date: Nov. 6, 2025

(54) METHOD FOR PREPARING CARBON NANOTUBE/SHAPE MEMORY POLYMER FOAM COMPOSITE, AND CATHETER SYSTEM USING AN ACTUATOR MADE OF CARBON NANOTUBE/SHAPE MEMORY POLYMER FOAM COMPOSITE

(71) Applicant: National Tsing Hua University, Hsinchu City (TW)

(72) Inventors: Wei-Chih WANG, Sammamish, WA (US); Yu-Yang HSU, Taoyuan City (TW); Fiona Marie WANG, Seattle, WA (US); Samuel RADOCHONSKI, Lafayette, CA (US)

(73) Assignee: National Tsing Hua University, Hsinchu City (TW)

(21) Appl. No.: 18/654,737

(22) Filed: May 3, 2024

(51) Int. Cl. C08K 3/04; A61M 25/00; A61M 25/01

(52) U.S. Cl. CPC C08K 3/041; A61M 25/0012; A61M 25/0054; A61M 25/01; A61M 2025/0058; A61M 2205/0216; A61M 2205/0266; A61M 2205/3368; A61M 2205/36; A61M 2205/50; A61M 2205/581; A61M 2205/582; A61M 2205/583; A61M 2207/10; C08K 2201/011

(57) ABSTRACT

A method for preparing a carbon nanotube/shape memory polymer (CNT/SMP) foam composite includes: performing a mixing process to mix a carbon nanotube (CNT) material, a shape memory polymer (SMP) material and a scaffolding material so as to obtain a CNT/SMP mixture which includes CNT/SMP nanocomposites and the scaffolding material; subjecting the CNT/SMP nanocomposites in the CNT/SMP mixture to a recrystallization process so as to obtain a recrystallized product in which the scaffolding material is trapped within recrystallized CNT/SMP nanocomposites; and after the recrystallization process, removing the scaffolding material from the recrystallized CNT/SMP nanocomposites.

### FIELD

The disclosure relates to a method for preparing a carbon nanotube/shape memory polymer foam composite, and more particularly to a method for preparing a carbon nanotube (CNT)/shape memory polymer (SMP) foam composite that is used for making an actuator of a catheter, and a catheter system that uses an actuator made of the CNT/SMP foam composite.

### BACKGROUND

Catheters are used in cardiovascular diseases to access heart through veins or arteries, and thus are desirable to have actuators with reduced diameters and small bending diameters. The industry is motivated to develop shape memory polymer (SMP)-based catheters because the configuration of SMP can be altered in response to a trigger, such as change of temperature, magnetic field or light, etc. SMP allows actuators of catheters made therefrom to have great flexibility with improved bending motions. Novel approaches for preparing more advanced SMP-based catheters are urged to further enhance the performance of SMP-based catheters.

### SUMMARY

Therefore, an object of the disclosure is to provide a method for preparing a carbon nanotube/shape memory polymer foam composite that can alleviate at least one of the drawbacks of the prior art.

According to the disclosure, the method for preparing a carbon nanotube/shape memory polymer (CNT/SMP) foam composite includes:
performing a mixing process to mix a carbon nanotube (CNT) material, a shape memory polymer (SMP) material and a scaffolding material so as to obtain a CNT/SMP mixture which includes CNT/SMP nanocomposites and the scaffolding material;
subjecting the CNT/SMP nanocomposites in the CNT/SMP mixture to a recrystallization process so as to obtain a recrystallized product in which the scaffolding material is trapped within recrystallized CNT/SMP nanocomposites; and
after the recrystallization process, removing the scaffolding material from the recrystallized CNT/SMP nanocomposites.

Another object of the disclosure is to provide a catheter system that uses the CNT/SMP foam composite of this disclosure in an actuator of a catheter.

According to the disclosure, the catheter system includes a catheter and a catheter controller. The catheter includes an actuator that is made of a CNT/SMP foam composite. The catheter controller is electrically connected to the actuator, and is operable to generate and send an electric driving signal to the actuator to heat the actuator to a temperature exceeding a turn-on temperature. The actuator is configured to, after being deformed into a temporary shape, remain in the temporary shape when the actuator is at a temperature lower than the turn-on temperature. The actuator is configured to automatically deform according to a predetermined shape when the actuator is at the temperature exceeding the turn-on temperature.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a flow chart illustrating a method for preparing a carbon nanotube/shape memory polymer (CNT/SMP) foam composite in accordance with some embodiments.

FIG. 2 is a flow chart illustrating a method to obtain a CNT/SMP mixture in accordance with some embodiments.

FIG. 3A is a photo illustrating a syringe barrel, a nozzle and a glass tube in accordance with some embodiments.

FIG. 3B is an enlarged schematic cross-sectional view illustrating communication among the syringe barrel, the nozzle and the glass tube in accordance with some embodiments.

FIG. 4A is a photo illustrating a plunger with a built-in needle in accordance with some embodiments.

FIG. 4B is an enlarged perspective view illustrating an end portion of the plunger from a different angle.

FIG. 5A is a photo illustrating an extrusion system in accordance with some embodiments

FIG. 5B is an enlarged schematic cross-sectional view illustrating a CNT/SMP mixture being extruded into water in accordance with some embodiments.

FIG. 6 is a schematic flow diagram illustrating formation of a cured product in accordance with some embodiments.

FIG. 7 is a schematic view illustrating removal of a scaffolding material from the cured product in accordance with some embodiments.

FIG. 8 is a photo illustrating (a) a cured product (on the left side) and (b) the CNT/SMP foam composite (on the right side) in accordance with some embodiments.

FIG. 9 is a photo of the CNT/SMP foam composite formed with a hole at the center thereof in accordance with some embodiments.

FIG. 10 is a schematic flow diagram illustrating formation of copper electrodes in accordance with some embodiments.

FIG. 11 is a photo of the CNT/SMP foam composite assembled with copper electrodes and wire in accordance with some embodiments.

FIG. 12 shows a heat distribution pattern of an actuator sample.

FIG. 13 shows a graph illustrating force required to bend the actuator sample to different bending displacements at 25° C. throughout different cycles.

FIG. 14 shows a graph illustrating resistance of the actuator sample after being bent at different bending displacements at 25° C. throughout different cycles.

FIG. 15 shows a graph illustrating the force required to bend the actuator sample to different bending displacements at 50° C. throughout different cycles.

FIG. 16 shows a graph illustrating the resistance of the actuator sample after being bent at different bending displacements at 50° C. throughout different cycles.

FIG. 17 shows a graph illustrating the force required to bend the actuator sample to different bending displacements at 80° C. throughout different cycles.

FIG. 18 shows a graph illustrating the resistance of the actuator sample after being bent at different bending displacements at 80° C. throughout different cycles.

FIG. 19 shows a spring constant of the CNT/SMP foam composite of the actuator sample at different temperatures.

FIG. 20 shows a graph illustrating a reaction force of the CNT/SMP foam composite of the actuator sample at different strains induced due to stretching of the CNT/SMP foam composite.

FIG. 21 shows a graph illustrating a capacitance of the CNT/SMP foam composite of the actuator sample at different induced strains.

FIG. 22 shows a graph illustrating an inductance of the CNT/SMP foam composite of the actuator sample at different induced strains.

FIG. 23 is a block diagram illustrating an embodiment of an actuator controller in accordance with some embodiments.

FIG. 24 is a flow chart illustrating steps of an embodiment of a method for getting an actuator ready for use.

FIG. 25 is a schematic diagram illustrating various types of deformation of the actuator.

FIG. 26 is a perspective view illustrating a mold used for programming the actuator.

FIG. 27 is a schematic diagram illustrating an example of how the actuator and the actuator controller are used in practice.

FIG. 28 is a schematic diagram illustrating a catheter system in accordance with some embodiments.

FIG. 29 is a perspective view illustrating a catheter driving mechanism of the catheter system in accordance with some embodiments.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

The present disclosure provides a method for preparing a carbon nanotube/shape memory polymer (CNT/SMP) foam composite that can be applied for making an actuator of a medical catheter. The CNT/SMP foam composite made of a SMP material is capable of inducing shape memory effect and being deformed in response to a change of temperature. In addition, the CNT/SMP foam composite made of the CNT material is electrically conducting, and is capable of self-heating when being connected to an external power source, resulting in a change of temperature.

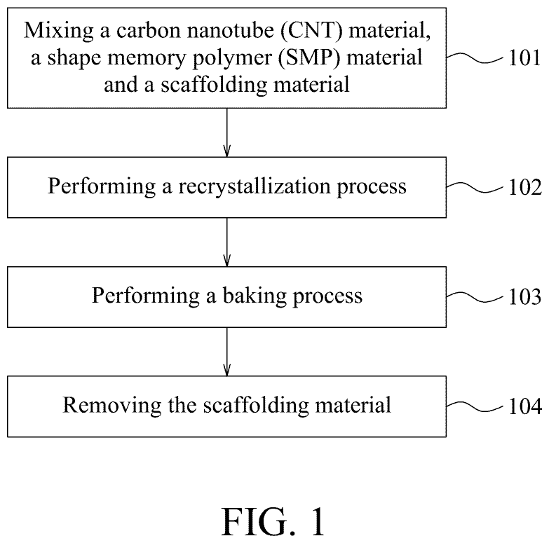

Referring to FIG. 1, the method includes the following steps 101 to 104.

In step 101, a mixing process is performed to mix a carbon nanotube (CNT) material, a shape memory polymer (SMP) material and a scaffolding material so as to obtain a CNT/SMP mixture which includes CNT/SMP nanocomposites and the scaffolding material.

The CNT material is configured to enhance electrical conductivity and mechanical properties of the CNT/SMP foam composite. In some embodiments, the CNT material includes single-walled carbon nanotubes (CNTs). The CNTs in the CNT material may have a diameter ranging from 50 nm to 90 nm. In some embodiments, carbon is present in an amount of greater than 95 wt % based on 100 wt % of the CNT material. In some embodiments, the CNT material is present in an amount ranging from 2 wt % to 5 wt %, such as 3.06 wt % to 3.19 wt %, based on 100 wt % of the CNT/SMP mixture.

The SMP material is configured to induce shape memory effect and deform in response to a trigger. In accordance with some embodiments of the present disclosure, the SMP material is a thermally induced SMP material, i.e., the SMP material deforms upon a change of temperature. Examples of the SMP material include polyurethane (PU), polytetrafluoroethylene (PFTE), polylactide (PLA), ethylene-vinyl acetate (EVA), amorphous polynorbornene, organic-inorganic hybrid polymers consisting of polynorbornene units that are partially substituted by polyhedral oligosilsesquioxane (POSS), epoxy resin (e.g., epoxy resin E-44, with a molecular weight of 450 g mol⁻¹ and epoxy equivalent weight of 210-240 g mol⁻¹), or poly(ethylene oxide)-poly(ethylene terephthalate) (PEO-PET) block copolymers that are crosslinked using maleic anhydride, glycerin or dimethyl 5-isophthalates. In some embodiments, PU is employed as the SMP material. Since PU is highly biocompatible, the CNT/CMP foam composite produced therefrom may be applied for making an actuator of a catheter which is to be used in a human body. In addition, PU is found to incorporate effectively with the CNT material. Other suitable thermally-induced SMP may also be used. In some embodiments, the SMP material is present in an amount ranging from 50 wt % to 80 wt %, such as 53.52 wt % to 71.28 wt %, based on 100 wt % of the CNT/SMP mixture.

The scaffolding material is configured to create pore structures within the CNT/CMP foam composite, which contributes to the CNT/CMP foam composite having a foamy structure, and thus permitting the CNT/CMP foam composite to have a high flexibility and a small bending angle. Specifically, the scaffolding material occupies spaces within the mixture throughout the process of forming the CNT/CMP foam composite, and is removed after solidification of the CNT/CMP foam composite to leave behind pore structures within the CNT/CMP foam composite. Specifically, the scaffolding material is inert and does not react with other components in the mixture. In addition, the scaffolding material may have a high melting point and does not melt until the CNT/CMP foam composite is formed, and thus remains intact and maintains the shape of the occupied spaces (that are to be formed into pore structures). That is, the size of the scaffolding material determines the size of the pore structures in the CNT/CMP foam composite. Moreover, the scaffolding material may be water soluble, such that after formation of the CNT/CMP foam composite, the scaffolding material can be readily dissolved in water and thus be removed by simply removing the water inside the CNT/CMP foam composite. In some embodiments, the scaffolding material may be a salt, such as sodium chloride, or the likes. In some embodiments, the scaffolding material is present in an amount ranging from 15 wt % to 25 wt %, such as 19.11 wt % to 19.95 wt %, based on 100 wt % of the CNT/SMP mixture.

In some embodiments, in the mixing process, a binder may be further added to be mixed with the CNT material, the SMP material and the scaffolding material so that the CNT/SMP mixture further includes the binder. The binder serves as a lubricant to uniformly blend and bind the CNT material, the SMP material, and the scaffolding material together. The binder may include an oil. Examples of the oil include olive oil, yellow bean oil, penetrating oil (e.g., WD-40®), combinations thereof, or the likes. In certain embodiments, the binder may be omitted. In some embodiments, the binder is present in an amount ranging from 0 wt % to 7 wt %, such as 0 wt % to 5.58 wt %, or 4.43 wt % to 5.58 wt %, based on 100 wt % of the CNT/SMP mixture.

In some embodiments, in the mixing process, a diluting agent (e.g., dimethylformamide (DMF) or other suitable materials) is further added to be mixed with the CNT material, the SMP material, the scaffolding material and the binder so that the CNT/SMP mixture further includes the diluting agent. The diluting agent is provided for diluting the SMP material, such that the CNT material can be easily and uniformly distributed among the SMP material. In certain embodiments, the diluting agent may be omitted. In some embodiments, the diluting agent is present in an amount ranging from 0 wt % to 20 wt %, such as 0 wt % to 19.88 wt %, based on 100 wt % of the CNT/SMP mixture.

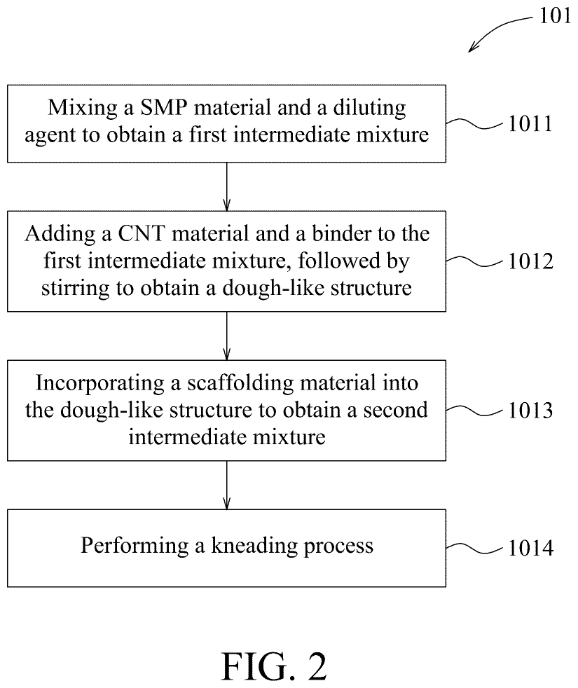

Referring to FIG. 2, in some embodiments, step 101 may include sub-steps 1011-1014 as follows.

Referring to FIG. 2, in sub-step 1011, a first intermediate mixture including the SMP material and the diluting agent is prepared. Specifically, the SMP material is weighed, and the diluting agent is weighed using a silicon beaker. The SMP material and the diluting agent are mixed and stirred until a mixture of the SMP material and the diluting agent achieves homogeneity, thereby obtaining the first intermediate mixture. In an exemplary embodiment, PU serves as the SMP material and DMF serves as the diluting agent. In certain embodiments, if the diluting agent is to be omitted, sub-step 1011 is also omitted.

Referring to FIG. 2, in sub-step 1012, the CNT material and the binder are weighed and added to the first intermediate mixture at the same time. The first intermediate mixture, the CNT material and the binder are stirred until a dough-like structure is obtained. The CNT material is uniformly dispersed in the dough-like structure. In an exemplary embodiment, single-walled CNTs serve as the CNT material, and an olive oil serves as the binder.

Referring to FIG. 2, in sub-step 1013, the scaffolding material is weighed and incorporated into the dough-like structure. The scaffolding material and the dough-like structure is stirred thoroughly to obtain a second intermediate mixture. In an exemplary embodiment, sodium chloride serves as the scaffolding material. In certain embodiments, before performing sub-step 1013, sodium chloride may be pre-processed first using a processor, so as to obtain sodium chloride in powdered form.

Referring to FIG. 2, in sub-step 1014, the second intermediate mixture is subjected to a kneading process, so as to allow each of the CNT material, the SMP material and the scaffolding material to be dispersed uniformly throughout the second intermediate mixture, thereby obtaining the CNT/SMP mixture. The CNT/SMP mixture includes the CNT/SMP nanocomposites formed from the CNT material and the SMP material, the scaffolding material, the binder (optional), and the diluting agent (optional).

Referring to FIG. 1, in step 102, the CNT/SMP nanocomposites in the CNT/SMP mixture are subjected to a recrystallization process to obtain a recrystallized product, in which the scaffolding material is trapped within recrystallized CNT/SMP nanocomposites.

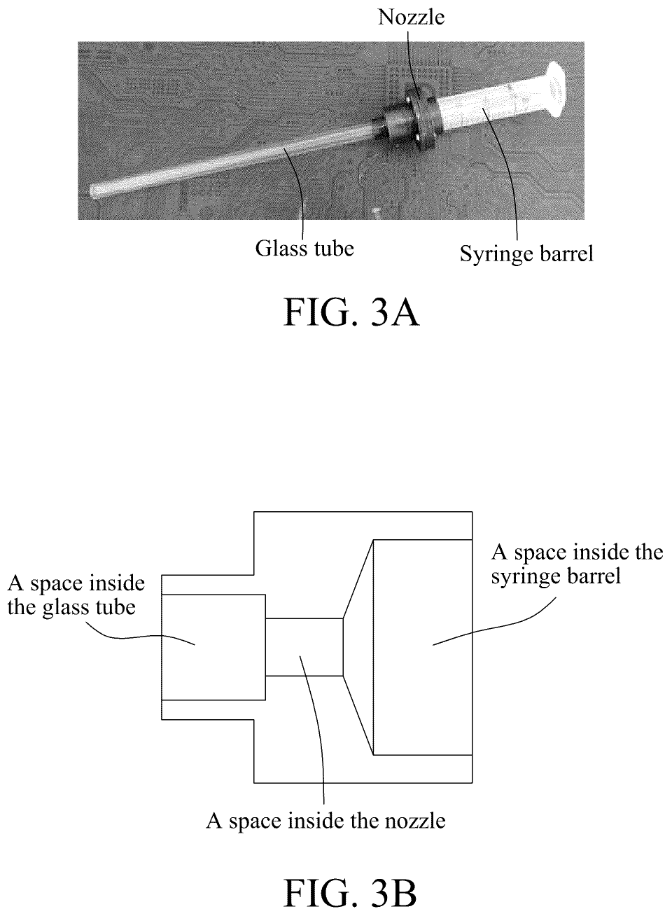

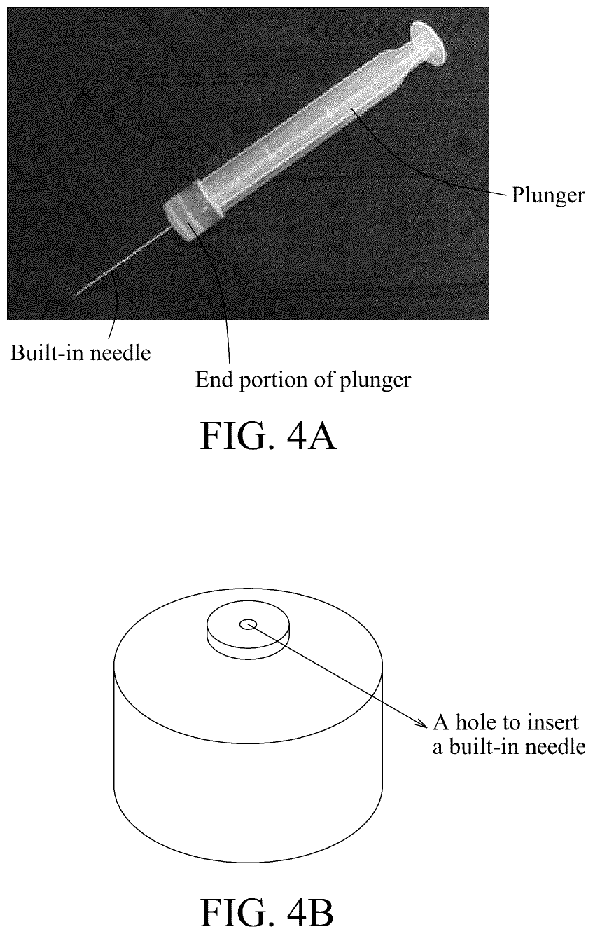

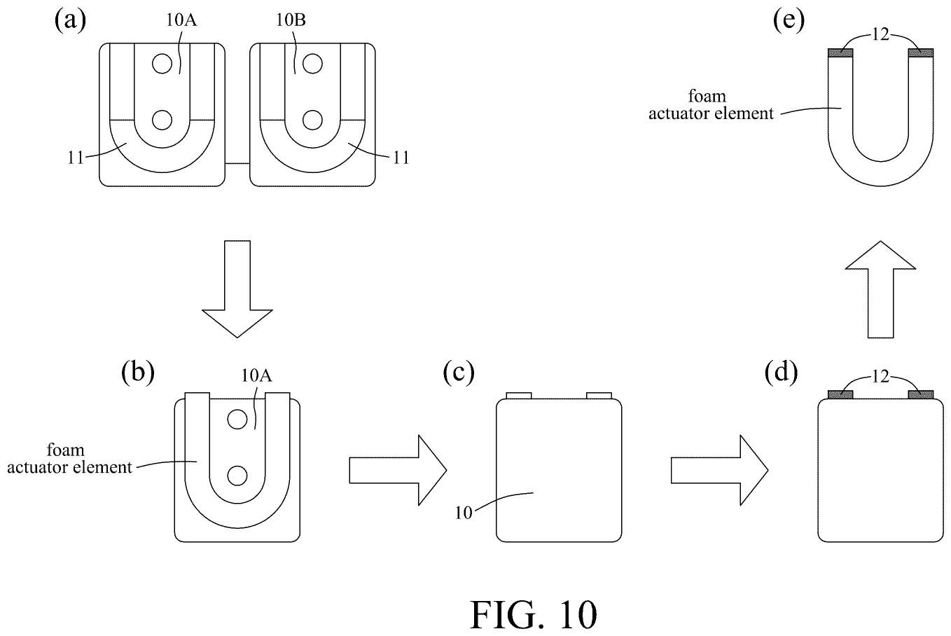

In step 102, the CNT/SMP mixture may be shaped using an extrusion system. The extrusion system includes a syringe pump, a plunger, a syringe barrel, a nozzle, and a glass tube (see FIGS. 3A and 3B). In some embodiments, the syringe barrel and the glass tube are detachably connected to two opposite ends of the nozzle, respectively. The CNT/SMP mixture is placed in the syringe barrel, and is then extruded into the glass tube through the nozzle using the plunger, so as to form the extruded CNT/SMP mixture in a cylinder shape. An inner diameter of the glass tube may be determined according to a desired outer diameter of the CNT/SMP foam composite. Please note that the mixture may shrink after the recrystallization process and other processes subsequent to the recrystallization process (e.g., a baking process which will be discussed later in step 103). For instance, the glass tube having an inner diameter of 8 mm may result in a CNT/SMP foam composite in a cylinder shape with an outer diameter of 5 mm. In certain embodiments, in order to form the CNT/SMP foam composite as a tube (with a hole at center of the CNT/SMP foam composite that extends along an extension direction of the CNT/SMP foam composite, see also FIG. 9), the plunger may include a built-in needle (see FIGS. 4A and 4B). The built-in needle may have an outer diameter of, e.g., 1 mm. In an exemplary embodiment, the nozzle has an inner diameter of 6 mm, the glass tube has an outer diameter of 10 mm, an inner diameter of 8 mm, and a length of 200 mm, and the built-in needle has an outer diameter of 1 mm.

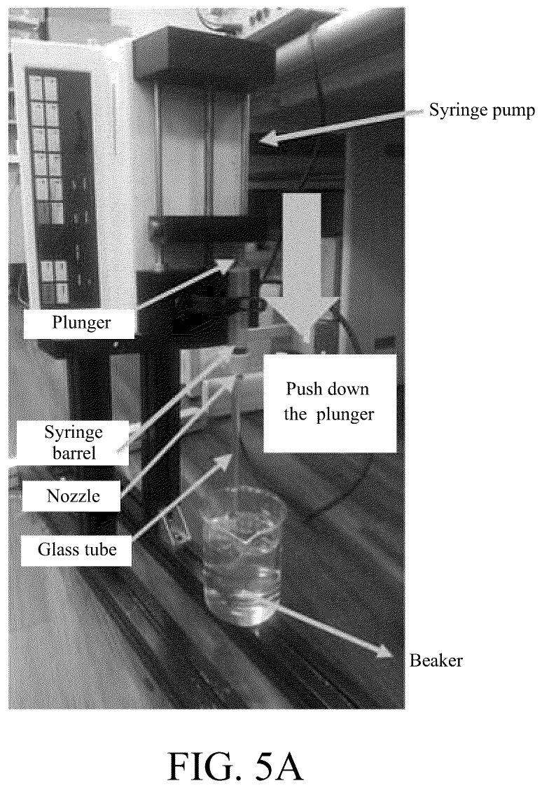

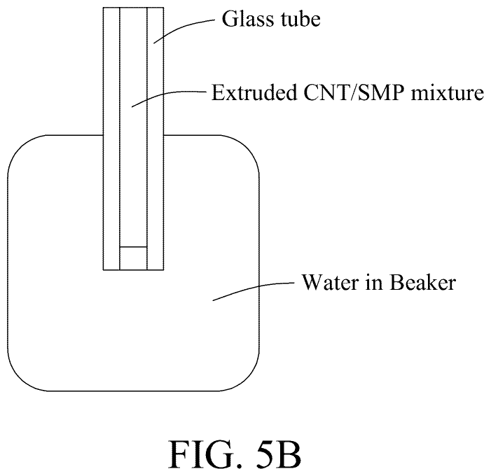

Referring to FIGS. 5A and 5B, the glass tube has one end connected to the nozzle, and an opposite end immersed in water contained in a 500 ml beaker at room temperature. In some embodiments, the water is deionized water (DI water). The syringe pump is configured to drive the plunger to extrude the CNT/SMP mixture, in a bubble-free manner at an optimal constant speed (e.g., 10 mL/minute, but not limited thereto), from the syringe barrel into the glass tube. The extruded CNT/SMP mixture is accommodated within the glass tube and is in contact with the water in the beaker.

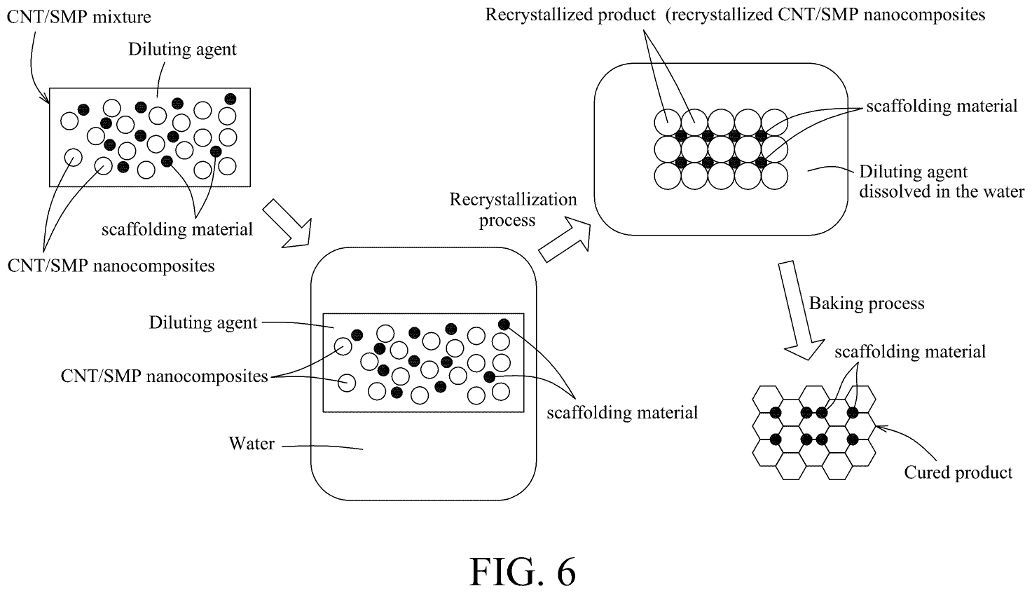

FIG. 6 is a flow diagram illustrating formation of a cured product in accordance with some embodiments. Referring to FIGS. 5A, 5B and 6, once the extruded CNT/SMP mixture is in contact with the water, the diluting agent (e.g., DMF) is readily dissolved in the water, and the binder (not shown in FIG. 6) leaves the recrystallized product and floats in the water. To be specific, after the extruded CNT/SMP mixture is brought into contact with the water to permit the CNT/SMP nanocomposites to be subjected to the recrystallization process, the CNT/SMP nanocomposites react with water and are rearranged into a more ordered structure (in comparison with the structure of the CNT/SMP nanocomposites in the extruded CNT/SMP mixture prior to being in contact with the water) with the scaffolding material being trapped within recrystallized CNT/SMP nanocomposites. In the case of powdered sodium chloride serving as the scaffolding material, it is noted that a majority of the powdered sodium chloride is trapped, and a minority of the powdered sodium chloride is dissolved in water. The recrystallized product is obtained after the recrystallization process.

Referring to FIG. 1, in step 103, the recrystallized product is subjected to a baking process, so as to cure and solidify the recrystallized product, thereby obtaining a cured product. In some embodiments, the baking process is conducted at a temperature of not greater than a programming temperature of the CNT/SMP foam composite (a permanent shape of the CNT/SMP foam composite is defined at the programming temperature), such as 140° C., but is not limited thereto. During step 103, the scaffolding material does not melt, and the shapes of the spaces occupied by the scaffolding material in the recrystallized CNT/SMP nanocomposites are maintained until the recrystallized product is formed into the cured product. In this case, a salt, e.g., sodium chloride, is an ideal candidate for the scaffolding material due to high melting point thereof.

Referring to FIG. 1, in step 104, the scaffolding material is removed from the cured product. In some embodiments, step 104 includes two sub-steps.

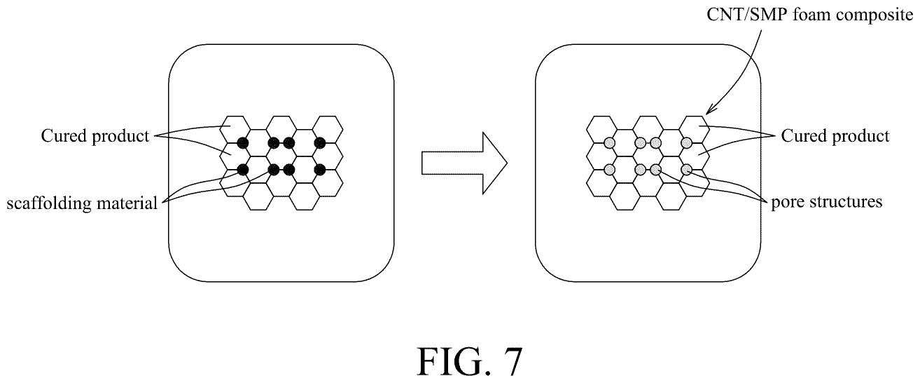

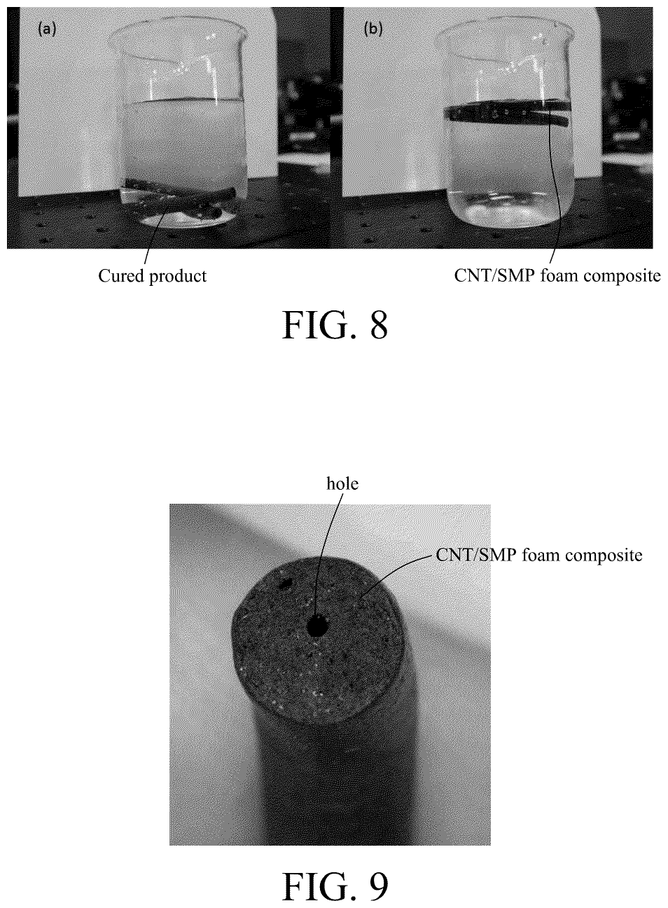

In the first sub-step of step 104, the cured product is first immersed in a liquid for dissolving the scaffolding material. In some embodiments, the liquid is hot water. The scaffolding material (see the black circles on the left side of FIG. 7) trapped in the cured product is readily dissolved in the hot water to leave pore structures (see the circles with lighter shade on the right side of FIG. 7), thereby obtaining the CNT/SMP foam composite. In some embodiments, the hot water may have a temperature of approximately 60° C., but is not limited thereto, as long as the scaffolding material is dissolved and removed. Before the scaffolding material is dissolved in the hot water, the cured product sinks to the bottom of a container of the hot water. After completion of the first sub-step of step 104, the CNT/SMP foam composite floats in the hot water. FIG. 8 is a photo illustrating (a) the cured product (before removing the scaffolding material), and (b) the CNT/SMP foam composite (after removing the scaffolding material).

In the second sub-step of step 104, another baking process is performed to remove any water remaining within the CNT/SMP foam composite. In some embodiments, the baking process may be performed at a temperature of approximately 120° C., but is not limited thereto, so as to completely dry the CNT/SMP foam composite. In some embodiments, after the second sub-step of step 104, the CNT/SMP foam composite may be formed, and in some cases, formed with a hole at the center (see FIG. 9).

### Assembling Electrodes and Wires

The CNT/SMP foam composite formed with the hole may serve as a foam actuator element. The foam actuator element is assembled with electrodes and wires for electrical conduction, so as to be used as an actuator of a catheter. Various electrodes may be applied, and some of the examples are described as follows.

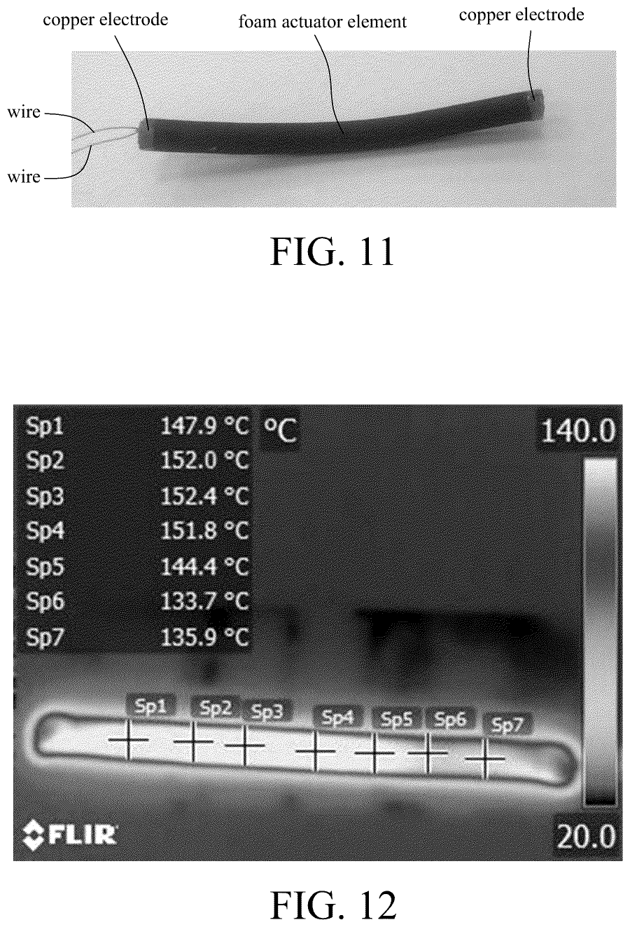

In accordance with some embodiments, copper electrodes are assembled at two opposite ends of the foam actuator element. FIG. 10 is a schematic flow diagram illustrating formation of forming copper electrodes in accordance with some embodiments. To be specific, two mold parts 10A, 10B each has a U-shape groove 11 as shown in part (a) of FIG. 10. After a half of a main part of the foam actuator element is disposed in the U-shape groove 11 of the mold part 10A as shown in part (b) of FIG. 10, the two opposite ends of the foam actuator element are disposed outwardly of the mold part 10A. Then, the mold part 10B is combined with the mold part 10A so that another half of the main part of the foam actuator element is received in the U-shape groove 11 of the mold part 10B with the two opposite ends of the foam actuator element exposed from an assembly 10 of the mold parts 10A, 10B as shown in part (c) of FIG. 10. Next, silver is sputtered toward the assembly 10, thereby forming two silver seed layers 12 respectively on the two opposite ends of the foam actuator element, as shown in part (d) of FIG. 10. Thereafter, the foam actuator element with the two silver seed layers 12 is removed from the assembly 10, as shown in part (e) of FIG. 10. A copper electroplating deposition process is then performed to form two copper electrodes (see FIG. 11) respectively on the silver seed layers 12. In some embodiments, two wires are connected respectively to the two copper electrodes to form an actuator of a catheter, where one of the wires is inserted into the hole of the foam actuator element (see FIG. 9) and penetrates the entire foam actuator element for connection to the respective copper electrode (e.g., the copper electrode at the right side in FIG. 11), so that external components (e.g., power sources) can be connected to the wires at the same side of the foam actuator element.

In accordance with some other embodiments, graphite electrodes are formed at the two opposite ends of the foam actuator element. Specifically, graphite powder is applied onto the two opposite ends of the foam actuator element under pressure. In some embodiments, similar to the case of copper electrode as described above, a wire is inserted in a hole of the foam actuator element that penetrates the entire length of the foam actuator element.

To evaluate the performance of the foam actuator element with the copper electrodes or with the graphite electrodes, six foam actuator element samples are formed with copper electrodes (denoted as C1, C2, C3, C4, C5, C6), and six foam actuator element samples are formed with graphite electrodes (denoted as G1, G2, G3, G4, G5, G6), and subjected to measurement of resistance. The results are shown in Table 1.

| | |
|---|---|
| Copper electrode | Graphite electrode |

| | | | | |
|---|---|---|---|---|
| Resistance | C1 | 78.978 | G1 | 112.1 |
| (ohm) | C2 | 98.841 | G2 | 101.77 |
| | C3 | 95.293 | G3 | 107.6 |
| | C4 | 127.74 | G4 | 103.24 |
| | C5 | 108.21 | G5 | 107.86 |
| | C6 | 116.58 | G6 | 144.95 |

| | | |
|---|---|---|
| Average | 104.27 | 112.92 |

resistance (ohm)

| | | |
|---|---|---|
| Standard | 17.14 | 16.12 |

deviation

It is noted that the samples formed with copper electrodes generally have lower resistance than the samples formed with graphite electrodes. It is noted that the samples formed with graphite electrodes are superior to the samples formed with the copper electrodes due to rapid production and reduced cost.

### Packaging and Electromagnetic Shielding

The foam actuator element may be applied as actuator of medical catheter to be used in a patient's body. In order to prevent current flow from the actuator to the patient's organ, the foam actuator element is packaged and sealed.

To package the foam actuator element that is assembled with the electrodes and wires, in some embodiments, an insulator coating is formed on the foam actuator element and the electrodes. The insulator coating prevents voltage shock, and is water proof. In some embodiments, the insulator coating includes an SMP material that is same as or different from the SMP material used in the CNT/SMP foam composite (and hence in the foam actuator element). The SMP material is an ideal material for forming the insulator coating not only because of its insulating property (capable of insulating electromagnetic energy), but also because of its shape memory property, such that the insulator coating does not hinder shape memory effect and/or shape recovery of the foam actuator element that operates as the actuator.

In some embodiments, the foam actuator element is dip-coated in a diluted SMP solution. The diluted SMP solution may include the SMP material and a solvent such as acetone, but is not limited thereto. In some embodiments, the SMP material may be present in an amount ranging from 40 wt % to about 50 wt %, such as 46.45 wt %, based on 100 wt % of the diluted SMP solution, and the solvent makes up the balance of the diluted SMP solution. The foam actuator element is first dip-coated with the diluted SMP solution, and is then placed in water to allow a curing process to proceed, followed by baking at approximately 60° C. to cure the diluted SMP solution, thereby obtaining the insulator coating. Before the curing process, the diluted SMP solution has a white color. During the baking process, the diluted SMP solution coated on the foam actuator element is cured and shrinks, turns shiny and transparent and eventually becomes the insulator coating. To confirm the functionality of the insulator coating, surface resistance and voltage leak, if any, the insulator coating may be measured using a multimeter (simulating operation of the actuator at DC 6 volts).

Evaluation of an Actuator Made from the CNT/SMP Foam Composite

### Heat Distribution Pattern

A uniform heat distribution throughout the CNT/SMP foam composite is important to avoid partial actuation, or overheating at certain region of the CNT/SMP foam composite when the CNT/SMP foam composite is made into an actuator.

A CNT/SMP foam composite sample was prepared in accordance with the method of the present disclosure, and was further processed to form an actuator sample, which was subjected to evaluation of heat distribution performance. A forward looking infrared (FLIR) camera was used to capture a heat distribution pattern of the actuator sample after the actuator sample had been activated for a certain period.

FIG. 12 shows the heat distribution pattern of the actuator sample. It is noted that there is merely little temperature deviation throughout the entire actuator sample. In a conventional SMP-based actuator, temperature deviation may be more than 20%. In contrast, the actuator sample using the CNT/SMP foam composite of the present disclosure may have a temperature deviation that is less than 15%. That is, the actuator sample has a remarkably uniform heat distribution pattern, and the highest concentration of heat is observed at the center of the actuator sample. It is noted that a CNT/SMP foam composite prepared by the method of the present disclosure, when connected to a power source for activation, shows a consistent and controlled heat distribution pattern.

### Mechanical and Sensing Test

The actuator sample is also subjected to a cyclic loading test to evaluate resistance change in response to temperature and displacement. The cyclic loading test is performed by cyclically bending and unbending the actuator sample to different bending displacement at a constant speed.

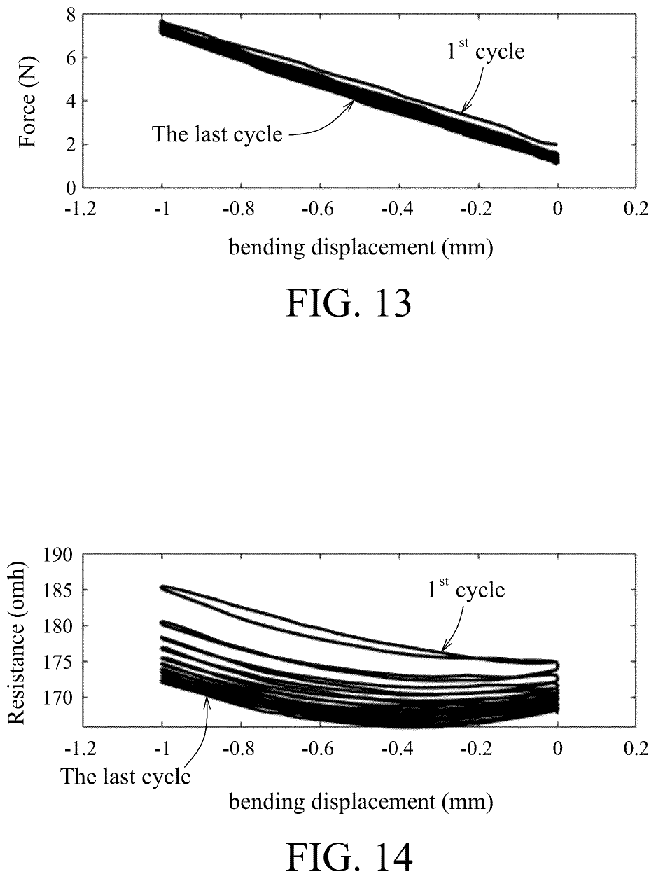

The cyclic loading test is first performed at 25° C. FIG. 13 shows a graph illustrating force (measured in terms of N) required to bend the actuator sample to different bending displacements (measured in terms of mm, bending displacement refers to a deformation of the actuator sample caused by a loading probe pushing thereon to bend the actuator sample) at 25° C., which indicates change of mechanical property of the CNT/SMP foam composite throughout the different cycles. FIG. 14 shows a graph illustrating resistance (measured in terms of ohm) of the actuator sample after being bent at different bending displacements at 25° C., which indicates change of electrical property of the CNT/SMP foam composite throughout the different cycles.

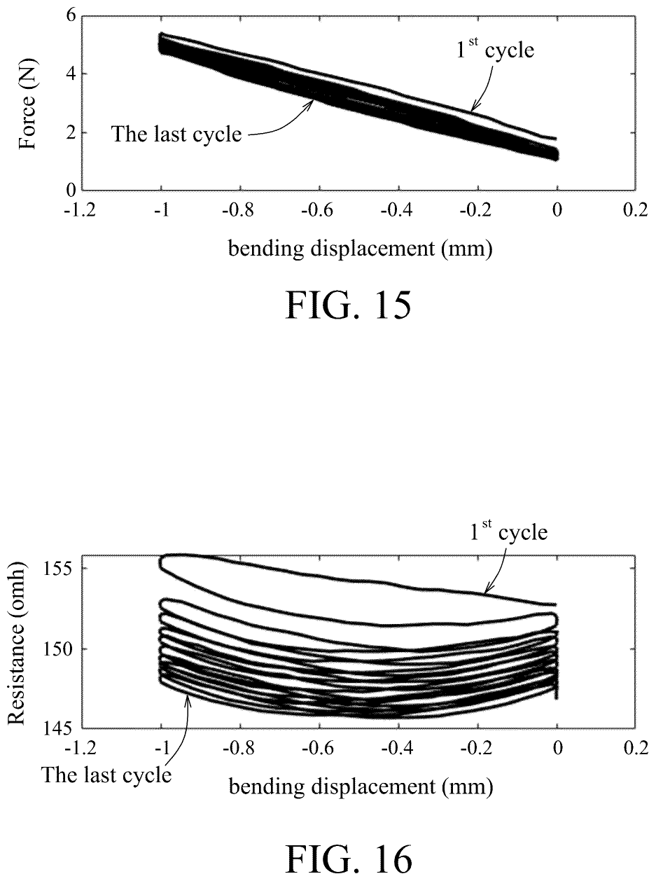

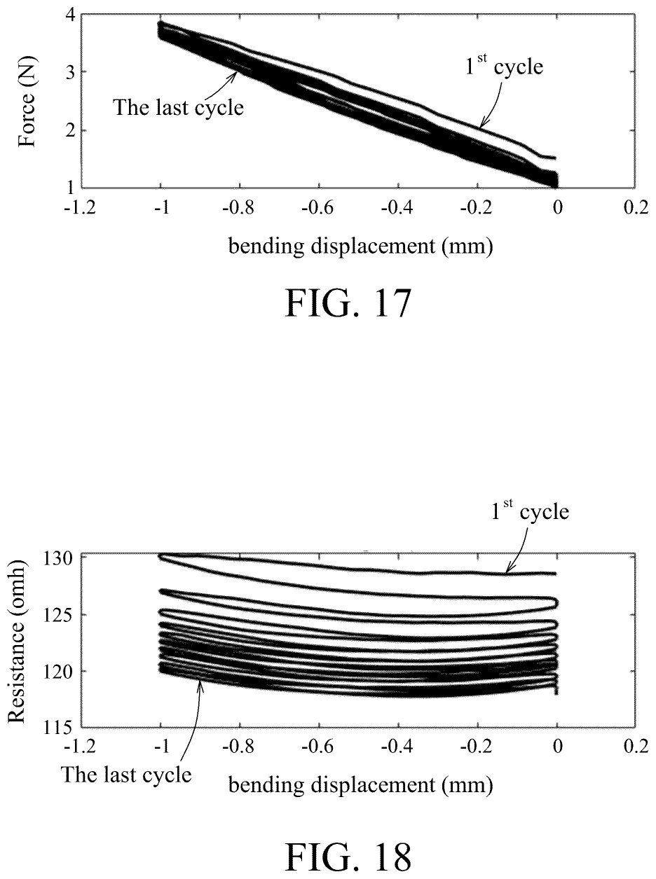

The cycling loading test is then repeated at 50° C. and 80° C. FIGS. 15 and 17 are graphs respectively illustrating the force required to bend the actuator sample to different bending displacements at 50° C. and at 80° C. FIGS. 16 and 18 are graphs respectively illustrating the resistance of the actuator sample after being bent at different bending displacements at 50° C. and at 80° C.

As shown in FIG. 13, the curves of the graph exhibit a mostly linear relationship between the force required and the bending displacements in each of the cycles. Similarly, as shown in FIG. 14, the curves exhibit a mostly linear relationship between resistance and the bending displacements starting from the second cycle. In addition, as shown by the graphs in FIGS. 13 and 14, as number of cycles increases, the curve slightly shifts downward, but the linearity is retained (and with similar slope). This result shows that the mechanical and electrical properties of the CNT/SMP foam composite are stable throughout cycles. The downward shifting could be attributed to polymer creep effects of the SMP material.

In addition, for cyclic loading test conducted at different temperatures, the curves of the graphs shown in FIGS. 15 and 17 show similar patterns as those of FIG. 13, and the curves of the graphs in FIGS. 16 and 18 show similar patterns as those of FIG. 14.

It is evident that mechanical and electrical performances of the CNT/SMP foam composite prepared using the method of the present disclosure are highly consistent at different temperatures. As such, the mechanical and electrical performances of the CNT/SMP foam composite prepared in accordance with the method of present disclosure are stable and do not change much over a certain period of time, or at different temperatures, and thus the CNT/SMP foam composite can ideally be used as an actuator of a medical catheter.

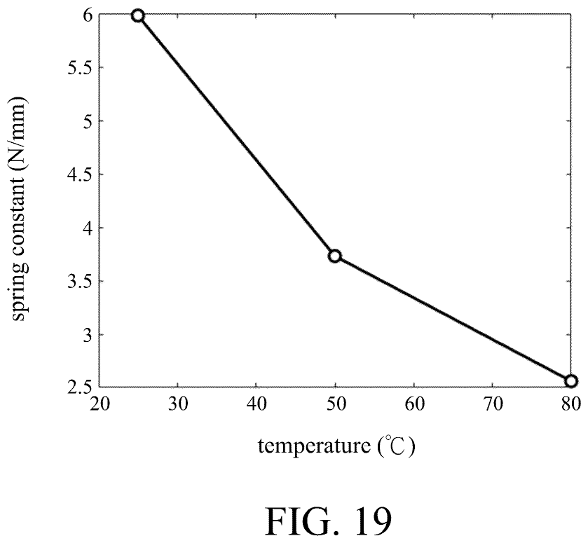

FIG. 19 shows a spring constant (measured in terms of N/mm) of the CNT/SMP foam composite of the actuator sample at different temperatures. It is noted that the spring constant decreases as temperature increases. Such decrement may be due to softening of the SMP material.

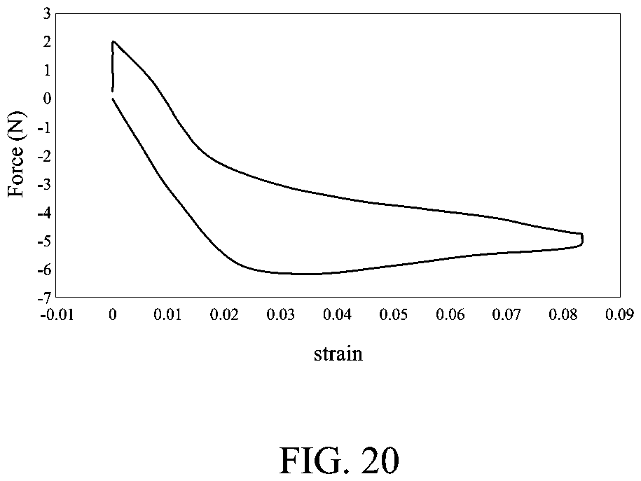

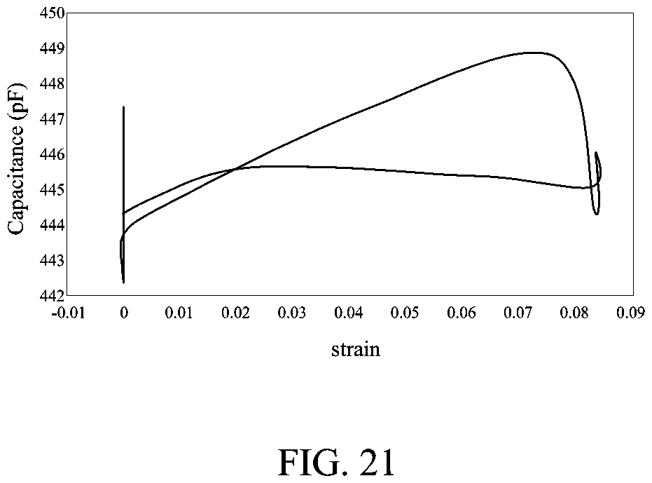

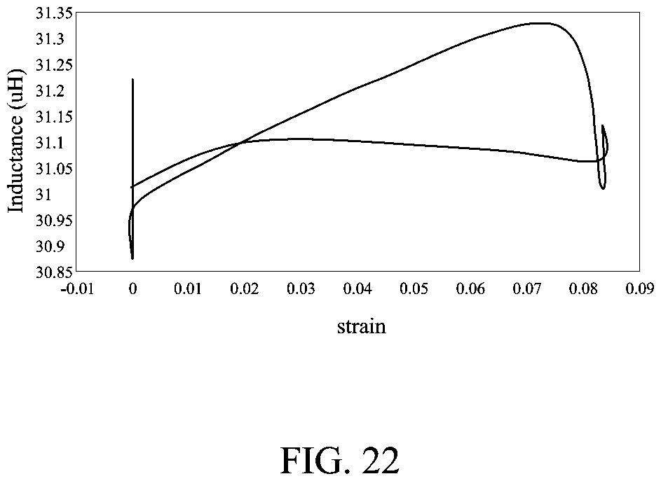

The actuator sample is then subjected to another assessment of electrical performances. The assessment is performed by applying 10 V to the actuator sample. The actuator sample is then activated (a current is induced to heat up the CNT/SMP foam composite of the actuator sample), and the CNT/SMP foam composite is subject to stretching, resulting in an elongated length Lₑ of the CNT/SMP foam composite, which is greater than an original length Lₑ of the CNT/SMP foam composite, and a strain (strain=(Lₑ−L₀)/L₀) is induced accordingly. A reaction force (measured in terms of N) due to the stretching of the CNT/SMP foam composite is measured. FIG. 20 shows a graph illustrating the reaction force of the CNT/SMP foam composite of the actuator sample at different induced strains. FIG. 21 shows a graph illustrating a capacitance (measured in terms of pF) of the CNT/SMP foam composite of the actuator sample at different induced strains. FIG. 22 shows a graph illustrating an inductance (measure din terms of pH) of the CNT/SMP foam composite of the actuator sample at different induced strains. It is noted that as the CNT/SMP foam composite is deformed (e.g., stretched) and induced with the strain, both the capacitance and the inductance change accordingly.

### Assembling Controller

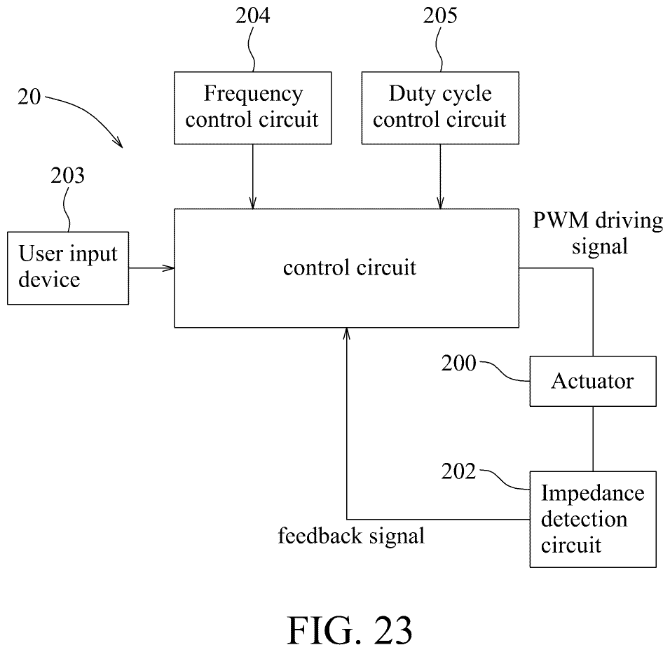

FIG. 23 illustrates an embodiment of an actuator controller 20 adapted to drive an actuator 200 according to this disclosure, where the actuator 200 is made of a CNT/SMP foam composite as introduced above, and has an impedance that varies based on a deformation condition thereof. The actuator controller 20 includes a control circuit 201, an impedance detection circuit 202, a user input device 203, a frequency control circuit 204, and a duty cycle control circuit 205.

The control circuit 201 is electrically connected to the actuator 200, and is configured to send a pulse width modulation (PWM) driving signal to the actuator 200, so as to heat up the CNT/SMP foam composite of the actuator 200 by electric current. In one example, the control circuit 201 may include a microcontroller capable of generating and outputting a PWM control signal, a level shifter to amplify the PWM control signal, and/or a drive circuit to output the amplified PWM control signal with sufficient power (namely, the PWM driving signal) to heat up the actuator 200.

The impedance detection circuit 202 is electrically connected to the actuator 200 and the control circuit 201, and is configured to sense the impedance of the actuator 200, and to generate a feedback signal that indicates the impedance of the actuator 200. In one embodiment, the impedance detection circuit 202 may include a voltage divider, an inductance bridge, a capacitance sensor, and the like, so the feedback signal is related to resistance, inductance and capacitance of the actuator 200 for the control circuit 201 to acquire the impedance of the actuator 200 accordingly. Then, the control circuit 201 may display the impedance of the actuator 200 on a display (not shown). Since the impedance of the actuator 200 would reflect the deformation condition (e.g., a bending/twisting angle, a level of contraction/extension, etc.) of the actuator 200, a user (e.g., a surgeon) may determine subsequent operation of the actuator controller 20 based on the impedance of the actuator 200.

The user input device 203 is communicatively connected to the control circuit 201 for the user to operate the control circuit 201, and may be realized as, for example, a button switch, a joystick controller, other suitable input devices, or any combination thereof.

The frequency control circuit 204 and the duty cycle control circuit 205 are used to set a frequency and a duty cycle of the PWM driving signal, thereby determining a heating speed of the actuator 200. The frequency control circuit 204 is electrically connected to the control circuit 201, and is configured to generate a frequency adjusting signal, so that the control circuit 201 adjusts a frequency of the PWM driving signal based on the frequency adjusting signal. In one example, the frequency control circuit 204 may include a variable resistor whose resistance may be adjusted by the user to adjust, for example, a magnitude of the frequency adjusting signal.

The duty cycle control circuit 205 is electrically connected to the control circuit 201, and is configured to generate a duty cycle adjusting signal, so that the control circuit 201 adjusts a duty cycle of the PWM driving signal based on the duty cycle adjusting signal. In one example, the duty cycle control circuit 205 may include a variable resistor whose resistance may be adjusted by the user to adjust, for example, a magnitude of the duty cycle adjusting signal.

### Using the Actuator

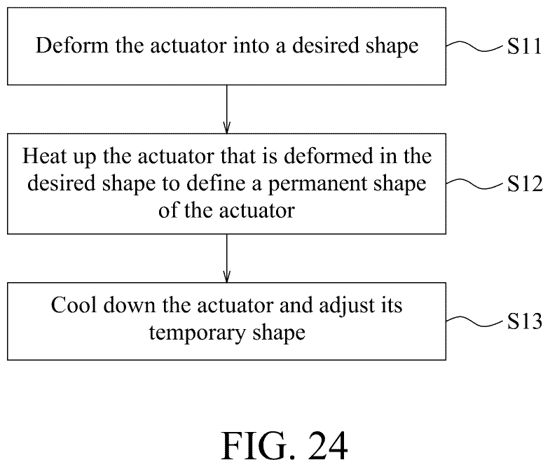

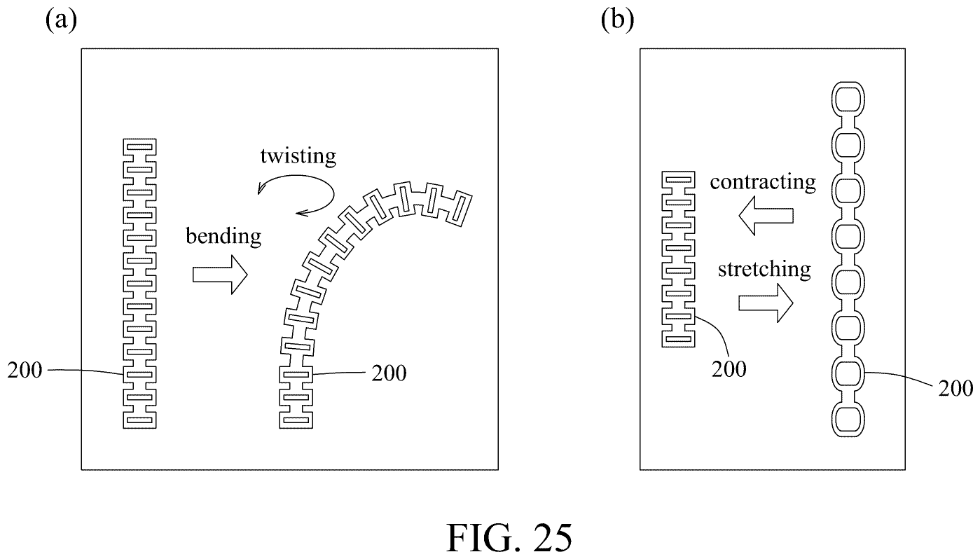

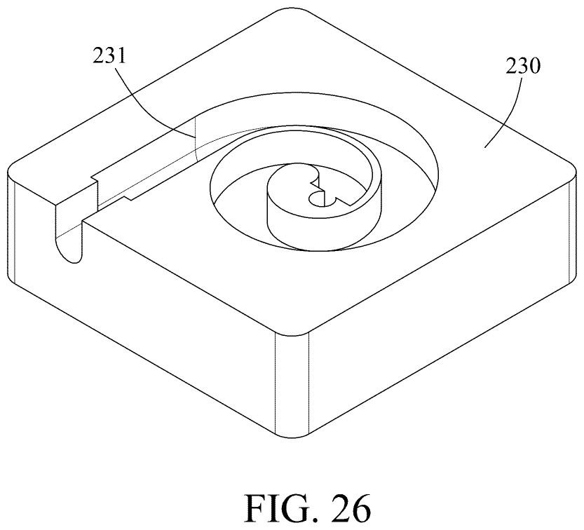

In order to make the actuator 200 capable of deforming as desired, the actuator 200 needs to be programmed. Further referring to FIG. 24, in step S11, an external force is exerted to deform the actuator 200 into a desired shape. The actuator 200 may be deformed by bending, twisting, stretching (to elongate the actuator 200), contracting (to shorten the actuator 200), etc., or any combination thereof, as illustrated in FIG. 25. In some embodiments, different sections of the actuator 200 may be deformed in different manners (e.g., different bending/twisting angles, different bending/twisting directions, different levels of stretching/contracting, etc.), so that the actuator 200 may have a snake-like shape. In one example, a mold may be prepared to have a recess in the desired shape for accommodating the actuator 200. FIG. 26 exemplarily illustrates a mold 230 patterned with a curved recess 231 for bending the actuator 200 into a spiral shape, but this disclosure is not limited in this respect. Then, the actuator 200 is heated up to a temperature over its programming temperature while being placed in the mold to define its permanent shape (step S12). In this embodiment, the aforesaid actuator controller 20 (see FIG. 23) can be used to program the actuator 200 by supplying electric current to heat up the CNT/SMP foam composite. Alternatively, an oven can also be used to program the actuator 200.

After completion of the programming, the actuator 200 is cooled down to, for example, room temperature. When the actuator 200 is at a temperature below a turn-on temperature, which is lower than the programming temperature and usually higher than the room temperature and body temperature of a human, the actuator 200 can be deformed from the permanent shape into any shape (referred to as temporary shape hereinafter) by an external force, and stay in the temporary shape when the external force is no longer applied. Accordingly, after the programming, the actuator 200 can be adjusted into a shape (step S13) that easily enters an artery (e.g., straightening the actuator 200) during surgery. Then, by heating up the actuator 200 to a temperature exceeding the turn-on temperature but lower than the programming temperature, the actuator 200 would automatically deform based on the permanent shape that has been programmed (e.g., having a tendency to revert to the permanent shape). In a case where the permanent shape of the actuator 200 is defined using the mold 230 as shown in FIG. 26, the actuator 200 may start to bend when being heated over the turn-on temperature. The speed of reversion may be influenced by the temperature; for example, the higher the temperature, the faster the reversion.

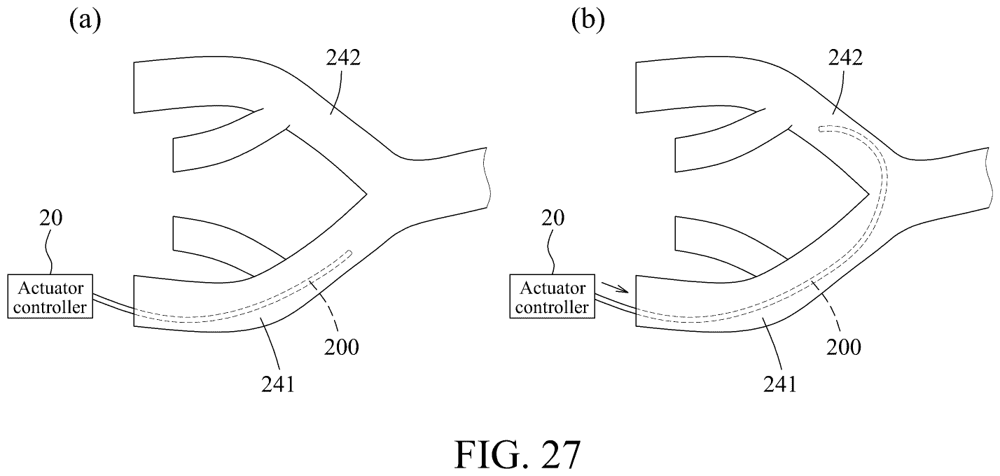

FIG. 27 is an example that illustrates how the actuator 200 is used in practice. In part (a), the actuator 200 had entered a femoral artery 241, and it was intended for the actuator 200 to enter another femoral artery 242. However, an angle the actuator 200 has to turn in order to enter the artery 242 from the artery 241 was so large that it would be difficult for the actuator 200 to enter the artery 242 in its current shape. Accordingly, as shown in part (b), the actuator controller 20 was operated to heat up the actuator 200 electrically to a temperature exceeding the turn-on temperature but lower than the programming temperature, thereby deforming the actuator 200. In this example, the actuator 200 was programmed to have a spiral permanent shape, so the actuator 200 bent in part (b) of FIG. 27, but this disclosure is not limited in this respect. In other embodiments, the deformation of the actuator 200 may be bending, twisting, stretching, contracting, other types of deformation, or any combination thereof, depending on how the actuator 200 was programmed. When the actuator 200 was deformed into a desired bending angle in part (b) of FIG. 27, the actuator controller 20 was operated to stop heating the actuator 200, thereby preventing the actuator 200 from further bending and maintaining the actuator 200 at the desired bending angle.

### Catheter System

In practice, a catheter often requires a considerable length to reach a specific target area from an artery. To accommodate this need, the actuator 200, which may be as short as illustrated in FIG. 11, would need to be connected with an additional wire to achieve the required length, such as one meter minimum. In one embodiment, a coaxial wire is soldered to the wires of the actuator 200 (see FIG. 11) to achieve the required length, and the control circuit 201 is electrically connected to the wires of the actuator 200 through the coaxial wire, but this disclosure is not limited to such.

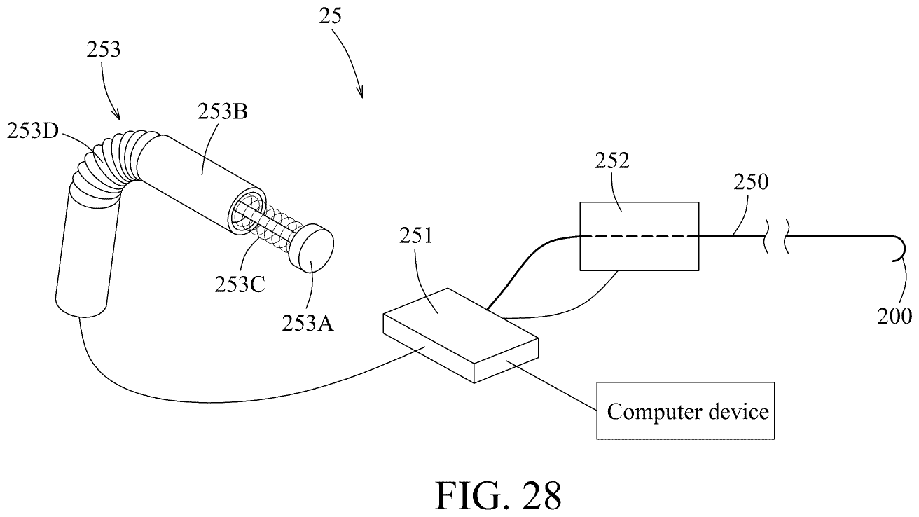

Referring to FIG. 28, an embodiment of a catheter system according to this disclosure is shown to include a catheter 250 and a catheter driving system 25. The catheter 250 includes the actuator 200 serving as a catheter tip. In some embodiments, the electrode at the end of the actuator 200 may be directly utilized for radio frequency (RF) ablation, but this disclosure is not limited in this respect. The catheter driving system 25 includes a catheter controller 251, a catheter driving mechanism 252 and a user operation device 253.

In one example, the catheter controller 251 may include the actuator controller 20 (see FIG. 23) that is electrically connected to the actuator 200. In one example, the control circuit 201 of the actuator controller 20 may be further electrically connected to the catheter driving mechanism 252 to control movement of the catheter 250 through the catheter driving mechanism 252, but this disclosure is not limited to such. In one example, the catheter controller 251 may include another control circuit to control operation of the catheter driving mechanism 252.

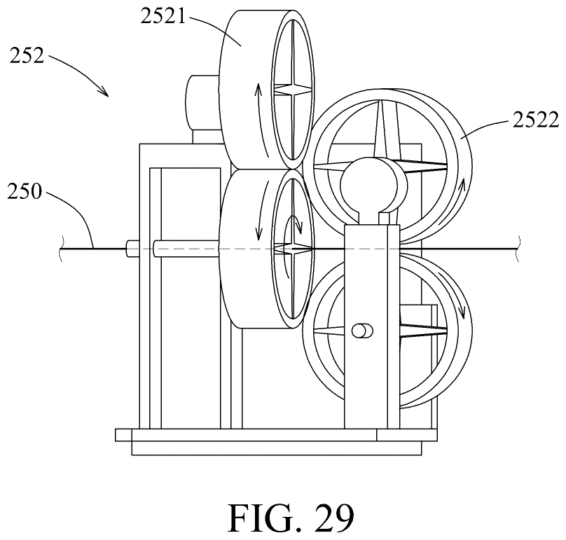

Further referring to FIG. 29, in one example, the catheter driving mechanism 252 may include a first wheel set 2521, a second wheel set 2522, and a motor module (not shown) that is connected to catheter controller 251 and the wheel sets 2521, 2522. In the illustrative embodiment, the catheter 250 penetrates the first wheel set 2521 through a center of a lower wheel of the first wheel set 2521, and is sandwiched between a pair of wheels of the second wheel set 2522. When the first wheel set 2521 is driven by the catheter controller 251 to rotate, the catheter 250 co-rotates with the lower wheel of the first wheel set 2521. When the second wheel set 2522 is driven by the catheter controller 251 to rotate, the catheter 251 would be fed forward or pulled backward according to rotational directions of the wheels of the second wheel set 2522, thereby achieving a threading operation.

The user operation device 253 is electrically connected to the catheter controller 251, and serves as the user input device 203 of the actuator controller 20 as illustrated in FIG. 23. In the illustrative embodiment, the user operation device 253 is a joystick controller that is configured to generate multiple types of operating signals in response to different user operations. For example, one type may be related to movement (e.g., rotation, moving forward, moving backward, etc.) of the catheter 250, and one type may be related to deformation (e.g., bending, twisting, stretching, contracting, etc.) of the actuator 200. The joystick controller includes a rotatable control stick 253A with a control head, a tube portion 253B, a spring 253C disposed between the control head and the tube portion 253B to define an initial position of the control stick 253A, and a flexible portion 253D connected to the tube portion 253B.

The user operation device 253 is configured to generate and send a movement signal (a first type of the operation signal) to the catheter controller 251 when the control stick 253A is operated to move in a user-desired manner (i.e., a way the user wants the catheter 250 to move in, such as rotating, moving forward, moving backward, etc.), and the catheter controller 251 controls the catheter driving mechanism 252 to move the catheter 250 in the user-desired manner based on the movement signal. To describe in further detail, when the control stick 253A is rotated, the joystick controller may send a rotation signal (the movement signal indicating rotation) to the catheter controller 251, so that the catheter controller 251 controls the first wheel set 2521 of the catheter driving mechanism 252 to rotate the catheter 250. When the control head of the control stick 253A is pushed toward the tube portion 253B against the spring 253C, the joystick controller may send a feeding signal (the movement signal indicating moving forward) to the catheter controller 251, so that the catheter controller 251 controls the wheels of the second wheel set 2522 of the catheter driving mechanism 252 to rotate in directions that make the catheter 250 extend forward; and once the control head of the control stick 253A is released, the spring 253C may bounce the control stick 253A back to the initial position. When the control head of the control stick 253A is pulled away from the tube portion 253B, the joystick controller may send a pulling signal (the movement signal indicating moving backward) to the catheter controller 20, so that the catheter controller 251 controls the wheels of the second wheel set 2522 of the catheter driving mechanism 252 to reverse their rotational directions to retract the catheter 250.

The flexible portion 253D may include an accordion-like structure that can be expanded and contracted. When the flexible portion 253D is deformed (e.g., bent, twisted, stretched, contracted, etc.) from a predefined shape (e.g., a linear shape), the joystick controller may send an actuating signal (a second type of the operating signal) to the catheter controller 251, so that the catheter controller 251 generates and sends the PWM driving signal based on the actuating signal to heat up and deform the actuator 200. In one embodiment, a speed of bending, twisting, stretching, and/or contracting the flexible portion 253D may be related to a magnitude, a frequency, and/or a duty cycle of the PWM driving signal that is sent to the actuator 200.

In some embodiments, the permanent shape of the actuator 200 is defined by bending the actuator 200, and the joystick controller is configured to generate and send the actuating signal to the catheter controller 251 when the flexible portion 253D is bent.

In some embodiments, the permanent shape of the actuator 200 is defined by twisting the actuator 200, and the joystick controller is configured to generate and send the actuating signal to the catheter controller 251 when the flexible portion 253D is twisted.

In some embodiments, the permanent shape of the actuator 200 is defined by stretching the actuator 200, and the joystick controller is configured to generate and send the actuating signal to the catheter controller 251 when the flexible portion 253D is stretched.

In some embodiments, the permanent shape of the actuator 200 is defined by contracting or compressing the actuator 200, and the joystick controller is configured to generate and send the actuating signal to the catheter controller 251 when the flexible portion 253D is contracted or compressed.

By virtue of the ergonomic joystick controller that mimics manual catheterization, the user may control the movement of the catheter 250 and the deformation of the actuator 200 intuitively, thereby promoting convenience of use and efficiency of surgical operations.

In some embodiments, the catheter controller 251 utilizes the impedance detection circuit 202 of the actuator controller 20 to provide a haptic feedback when the actuator 200 touches an object (e.g., a vascular wall) and deforms. For example, the catheter controller 251 is configured to send a user-perceivable signal when a change in the impedance of the actuator 200 as sensed by the impedance detection circuit 202 is greater than a predetermined criterion, so the user would be aware of the deformation of the actuator 200. In some embodiments, the catheter controller 251 is configured to derive the deformation condition of the actuator 200 based on the feedback signal provided by the impedance detection circuit 202 and the predetermined criterion that is related to the resistance, inductance and capacitance of the actuator 200, and to send the user-perceivable signal that reflects the deformation condition of the actuator 200 thus derived. The user-perceivable signal may be a visual signal, an audio signal, a tactile signal, or other types of signals that are perceivable by the user, or any combination thereof. In a case where the user-perceivable signal is a visual signal, the catheter controller 251 may be connected to, for example, light emitting diodes, and send the user-perceivable signal by controlling the light emitting diodes to emit flashing light that serves as the user-perceivable signal. In a case where the user-perceivable signal is an audio signal, the catheter controller 251 may be connected to, for example, a speaker, and control the speaker to output sound or audio messages that serves as the user-perceivable signal. In a case where the user-perceivable signal is a tactile signal, the catheter controller 251 may be connected to, for example, a vibrator, and control the vibrator to vibrate in a predefined manner that serves as the user-perceivable signal. In some embodiments, the catheter controller 251 may be connected to a computer device, and transmit information of the impedance of the actuator 200 as sensed by the impedance detection circuit 202 to the computer device. The computer device may assess the deformation condition of the actuator 200 based on the information of the impedance of the actuator 200, and perceivably output the deformation condition of the actuator 200 thus assessed using, for example, a display of the computer device.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for preparing a carbon nanotube/shape memory polymer (CNT/SMP) foam composite, comprising:
performing a mixing process to mix a carbon nanotube (CNT) material, a shape memory polymer (SMP) material and a scaffolding material so as to obtain a CNT/SMP mixture which includes CNT/SMP nanocomposites and the scaffolding material;
subjecting the CNT/SMP nanocomposites in the CNT/SMP mixture to a recrystallization process so as to obtain a recrystallized product in which the scaffolding material is trapped within recrystallized CNT/SMP nanocomposites; and
after the recrystallization process, removing the scaffolding material from the recrystallized CNT/SMP nanocomposites.

2. The method as claimed in claim 1, wherein in the mixing process, a binder is further mixed with the CNT material, the SMP material and the scaffolding material so that the CNT/SMP mixture further includes the binder.

3. The method as claimed in claim 2, wherein the binder is an oil selected from olive oil, yellow bean oil, penetrating oil, and combinations thereof.

4. The method as claimed in claim 2, wherein the mixing process includes:
adding the CNT material and the binder to the SMP material to obtain a dough-like structure;
incorporating the scaffolding material into the dough-like structure to form a mixture of the scaffolding material and the dough-like structure; and
subjecting the mixture of the scaffolding material and the dough-like structure to a kneading process.

5. The method as claimed in claim 1, wherein the recrystallization process includes bringing the CNT/SMP mixture into contact with water.

6. The method as claimed in claim 1, further comprising, after the recrystallization process, performing a baking process on the recrystallized product.

7. The method as claimed in claim 4, wherein the baking process is performed at a temperature of not greater than a programming temperature of the CNT/SMP foam composite.

8. The method as claimed in claim 1, wherein the scaffolding material includes a salt.

9. The method as claimed in claim 8, wherein the salt is sodium chloride.

10. The method as claimed in claim 1, wherein removing the scaffolding material includes immersing the recrystallized product in a liquid for dissolving the scaffolding material.

11. The method as claimed in claim 1, wherein the CNT material includes single-walled carbon nanotubes.

12. The method as claimed in claim 1, wherein the SMP material is a polyurethane.

13. A catheter system, comprising:
a catheter including an actuator that is made of a carbon nanotube/shape memory polymer (CNT/SMP) foam composite; and
a catheter controller electrically connected to said actuator, and operable to generate and send an electric driving signal to said actuator for heating said actuator to a temperature that exceeds a turn-on temperature;
wherein said actuator is configured to, after being deformed into a temporary shape, remain in the temporary shape when said actuator is at a temperature lower than the turn-on temperature; and
wherein said actuator is configured to automatically deform according to a predetermined shape when said actuator is at the temperature exceeding the turn-on temperature.

14. The catheter system as claimed in claim 13, wherein the electric driving signal sent by said catheter controller is a pulse width modulation (PWM) signal, and said actuator is operable to adjust at least one of a frequency or a duty cycle of the PWM signal, so as to adjust a speed of deformation of said actuator.

15. The catheter system as claimed in claim 13, further comprising a user operation device electrically connected to said catheter controller;
wherein said user operation device includes a flexible portion, and is configured to generate and send an actuating signal to said catheter controller when said flexible portion is deformed; and
wherein said catheter controller is configured to generate and send the electric driving signal to said actuator based on the actuating signal.

16. The catheter system as claimed in claim 15, further comprising a catheter driving mechanism electrically connected to said catheter controller, connected to said catheter, and operable to drive movement of said catheter;
wherein said user operation device includes a control stick, and is configured to generate and send a movement signal to said catheter controller when said control stick is operated to move in a user-desired manner; and
wherein said catheter controller is configured to control said catheter driving mechanism to move said catheter in the user-desired manner based on the movement signal.

17. The catheter system as claimed in claim 15, wherein the predetermined shape is defined by deforming said actuator through at least one of bending, twisting, stretching or contracting;
wherein said user operation device is configured to, when the predetermined shape is defined by bending said actuator, generate and send the actuating signal to said catheter controller when the flexible portion is bent;
wherein said user operation device is configured to, when the predetermined shape is defined by twisting said actuator, generate and send the actuating signal to said catheter controller when the flexible portion is twisted;
wherein said user operation device is configured to, when the predetermined shape is defined by stretching said actuator, generate and send the actuating signal to said catheter controller when the flexible portion is stretched; and
wherein said user operation device is configured to, when the predetermined shape is defined by contracting said actuator, generate and send the actuating signal to said catheter controller when the flexible portion is contracted.

18. The catheter system as claimed in claim 13, wherein said actuator is configured to have an impedance that varies based on a deformation condition of said actuator.

19. The catheter system as claimed in claim 18, wherein said catheter controller includes an impedance detection circuit configured to sense the impedance of said actuator; and
wherein said catheter controller is configured to send a user-perceivable signal when a change of the impedance of said actuator is greater than a predetermined criterion.

20. The catheter system as claimed in claim 18, further comprising a computer device, wherein said catheter controller includes an impedance detection circuit configured to sense the impedance of said actuator, and is configured to transmit information of the impedance of said actuator to said computer device; and
wherein said computer device is configured to assess the deformation condition of said actuator based on the information of the impedance of said actuator, and to perceivably output the deformation condition of said actuator thus assessed.
